(19) United States
(12) Patent Application Publication
(10) Pub. No.: US 2002/0072100 A1
(43) Pub. Date: Jun. 13, 2002

(54) Method to separate ethanol from a fermentation broth

(76) Inventor: Gene E. Lightner, Federal Way, WA (US)

Correspondence Address: Gene E. Lightner, 706 S.W. 296th St., Federal Way, WA, 98023-3549, US

(21) Appl. No.: 09/734,488

(22) Filed: Dec. 11, 2000

(51) Int. Cl. C12P 7/06

(52) U.S. Cl. 435/161

(57) ABSTRACT

This is a method to withdraw ethanol from a broth contained within a fermentation vessel. Fermentation broth contains sugars, microorganisms and nutrients maintained at a pH and temperature to influence rate of fermentation to form ethanol. Factors within the broth that also effect rate of fermentation are; concentration of sugars, activity of microorganisms and enzymes. These factors are controlled by addition of a mixture containing these constituents followed by removal of sludge and broth to substantially regulate broth volume within the vessel. Fermentation produces heat which must be removed from the broth in order to continue fermentation. Carbon dioxide, provided to the fermentation vessel, evaporates ethanol within the broth, by heat from fermentation, to humidify the carbon dioxide and accordingly regulate ethanol concentration of the broth. Withdrawal of ethanol from the broth achieves ethanol concentration of about 6% to about 12% within the broth. The method employs carbon dioxide, supplied to the fermentation vessel, to humidify ethanol and withdraw ethanol and carbon dioxide formed within the fermentation vessel. The carbon dioxide containing humidified ethanol is removed from the fermentation vessel. The removed ethanol humidified carbon dioxide, containing carbon dioxide produced from fermentation, is then substantially separated from the ethanol, and provides carbon dioxide for recycling to humidify additional ethanol. Broth and sludge, removed from the fermentation vessel, are transformed to substantially separate sludge from broth containing sugars.

### BACKGROUND OF THE INVENTION

Throughout the world there is increasing interest in converting renewable biomass to usable products such as ethanol.

Biomass contains two basic constituents, carbohydrates and lignin. The carbohydrate content of the biomass contains cellulose and hemicellulose. Both cellulose and hemicellulose may be converted to sugars of glucose and xylose. Fermentation converts glucose and xylose to ethanol using enzymes produced by microorganisms revealed in U.S. Pat. No. 5,789,210. Control of nutrients, pH, temperature, sugar concentration, and microorganism concentration all affect rate of fermentation to form ethanol. When ethanol concentration reaches above about 6 to 12%, ethanol concentration is lethal to the microorganisms employed for fermentation. To reduce ethanol concentration within broth employed for fermentation and maintain activity of microorganisms, extraction of ethanol from the broth by solvents non-toxic to microorganisms, is disclosed in U.S. Pat. Nos. 5,110,319, 4,865,973 and No. 4,517,298. The operations disclosed require energy for vaporization of ethanol and subsequent condensation to produce liquid ethanol.

It is therefore an object of this invention to obviate many of the limitations or disadvantages of the prior art.

The present concern of this invention is to prevent concentration of ethanol within a broth from reaching a concentration of ethanol lethal to microorganisms employed for fermentation.

An object of this invention is to remove ethanol contained within a broth by providing carbon dioxide to the broth to humidifying ethanol within the carbon dioxide.

An additional object is to substantially remove ethanol from ethanol humidified by carbon dioxide and provide carbon dioxide for subsequent humidification of ethanol.

Still another object of this invention is to vaporize ethanol within broth from heat from fermentation

Yet another object of this invention is to substantially maintain sugars and microorganisms within the fermentation vessel as required to continue fermentation.

Additionally an object of this invention is to substantially maintain volume of broth within the fermentation vessel

With the above and other objects in view, this invention relates to the novel features and alternatives and combinations presently described in the brief description of the invention.

### THEORETICAL BACKGROUND OF THE INVENTION

The principles applied herein employ Dalton's law and Raoult's law. Dalton's law of partial pressure may be expressed mathematically as P=p_{A}+p_{B}+ where p_{A} and p_{B} are the partial pressures of vapors A and B respectively and P is the total pressure. For only A and B, P=p_{A}+p_{B}, and the mole ratio of B to A is p_{B}/p_{A}=p_{B}/P−p_{B}. The weight ratio of A/B is p_{B}/P−p_{B} (molecular weight of B)/( average molecular weight of P−p_{B}). This is the equation used for humidity calculations when A is a gas and B is the vapor humidified. Raoult's law of partial pressure may be expressed mathematically as p solvent=p° solvent (N) solvent where p solvent is the partial vapor pressure of the solvent, p° solvent=the vapor pressure of the solvent times the mole fraction N of the solvent within a solution. Applying Raoult's law, let N=0.1 (the mole fraction of ethanol within a fermentation broth) and p° ethanol at a temperature Of 100° F., taking the partial vapor pressure at about 2.5 psia then p ethanol=0.1 (2.5) psia=0.25 psia.

The molecular weight of ethanol=46 and carbon dioxide has a molecular weight=44.

Applying the equation used for humidity, and let P=15 psia for a total pressure of humidified Carbon Dioxide, the weight ratio of ethanol/carbon dioxide is 0.25/15−0.25 (46/44)=0.018 lb. of ethanol/lb. of carbon dioxide. Accordingly a fermentation broth can have ethanol transferred by co-mingling carbon dioxide with the fermentation broth to form carbon dioxide humidified with ethanol. Humidified carbon dioxide by ethanol from fermentation is scrubbed by water to produce carbon dioxide substantially free of ethanol and water containing dissolved ethanol, disclosed by R. N. Shreve, Chemical Process Industries, 1956, page 130.

Raoult's law predicts that any volatile compound within a fermentation broth will form a partial vapor pressure of the volatile compound depending on the vapor pressure and mole fraction of the volatile compound within the fermentation broth. The equation used for humidity allows,that when a gas is humidified, the humidified gas may contain any partial vapor pressure of a volatile compound. Thus if the humidified carbon dioxide contains a partial vapor pressure of a volatile compound contained within the fermentation broth of the same partial vapor pressure of the same volatile compound then further humidification of the volatile compound such as water will not occur. Carbon dioxide, saturated within water, forms carbonic acid of pH level about 4; Therefore broth during fermentation is substantially maintained at a constant pH level. The reverse of humidification is dehumidification. These procedures occur with simultaneous heat and mass transfer. Humidification of ethanol to provide a vapor within a gas requires heat of vaporization derived from heat formed during fermentation of sugars. Sugars, capable of fermentation within which ethanol and carbon dioxide are produced, are selected from the group consisting of glucose, xylose and mixtures thereof Dehumidification of ethanol transfers ethanol vapors from a gas to a phase of a difference in ethanol partial pressure acquiring heat of vaporization of ethanol within the process to provide heat to the phase and consequently the sensible heat of the phase. Fermentation evolves heat as disclosed by R. N. Shreve, op.cit. pages 672-673. Ethanol vapor required to humidify ethanol is accordingly supplied by heat evolved during fermentation.

Microorganisms contained within broth will ultimately lose activity for fermentation and must be removed and replaced by active microorganisms. Enzymes produced from microorganisms are proteins that can be coagulated and precipitated by heat or chemical compounds as established by Hill and Kelley within Organic Chemistry, 1943, pages 442-443. Therefore broth containing diminish activity of microorganisms and enzymes can be heated to produce insoluble sludge within broth.

A ternary system created by benzene to form a low boiling point azeotrope with ethanol, water and benzene is employed within distillation columns to produce anhydrous ethanol as described by R. N. Shreve, op. cit., page 679. Analogously gasoline, ethanol and water form a low boiling point azeotrope which is utilized within distillation columns to produce anhydrous gasoline containing ethanol. Hydrocarbon compounds often found within gasoline are heptane and hexane. Azeotropes of these hydrocarbons, ethanol and water are listed in Handbook of Chemistry and Physics, 56th Edition, page D-42.

Salts subjected to water to form hydrates include calcium sulfate and aluminum sulfate as disclosed by R. N. Shreve, op.cit. page, 218 and page 436. Accordingly gasoline containing ethanol and water can be employed to produce anhydrous gasoline by forming a hydrate within the gasoline containing water followed by separation of the hydrate to yield anhydrous gasohol. For additional information, review F. Daniels, Outlines of Physical Chemistry and G. G. Brown, et al., Unit Operations.

### BRIEF DESCRIPTION OF THE INVENTION

The present invention in its broadest aspect, provides a method to withdraw ethanol from a fermented broth contained within a fermentation vessel. The preferred embodiment of the method employs carbon dioxide, supplied to the fermentation vessel, to humidify ethanol. Ethanol from the broth is transmitted to and co-mingled with carbon dioxide to humidify the carbon dioxide. The carbon dioxide containing humidified ethanol and carbon dioxide produced by fermentation is removed from the fermentation vessel and substantially separated from the ethanol. Carbon dioxide, substantially separated from the ethanol, is then purged of carbon dioxide to substantially equal carbon dioxide formed from fermentation. The carbon dioxide is then recycled to humidify additional ethanol within fermented broth. Consequently the fermented broth provides ethanol to humidifiy carbon dioxide which is then separated from the fermentation vessel.

Characteristics of the invention include;

A fermentation vessel is provided for fermentation of sugars to form ethanol and carbon dioxide from a fermentation broth contained within a fermentation vessel.

Carbon dioxide is provided to the fermentation vessel and co-mingled with the fermentation broth.

Ethanol and other volatile components contained within the fermentation broth are humidified by carbon dioxide and removed from the fermentation vessel. Depending on the composition of the fermented broth, volatiles contained within the carbon dioxide can be of several types including aldehydes, alcohols, esters and acids.. Carbon dioxide and ethanol produced from fermentation is substantially removed from the carbon dioxide.

Fermentation produces heat, that is substantially proportional to ethanol produced, that is employed to evaporate ethanol.

Microorganisms, required for fermentation, are replenished within the fermentation vessel to maintain their activity.

Sludge, sugars, nutrients and microorganisms are removed from the fermentation vessel as required to maintain the volume of the fermentation broth.

Withdrawal of ethanol from fermentation broth to humidify carbon dioxide is utilized to produce an ethanol concentration within the broth of about 6% to about 12%.

### BRIEF DESCRIPTION OF THE DRAWINGS

The features that are considered characteristic of this invention are set forth within the appended claims. This invention, however, both as to its origination and method of operations as well as additional advantages will best be understood from the following description when read in conjunction with the accompanying drawings in which:

FIG. 1 is a flow sheet denoting the invention as set forth in the appended claims.

FIG. 2 is a flow sheet denoting an alternate method for substantially separating ethanol from ethanol humidified carbon dioxide.

FIG. 3 is a flow sheet denoting a method to substantially separate sludge from broth contained within broth and sludge.

FIG. 4 is a flow sheet denoting a method to substantially absorb ethanol humidified carbon dioxide with gasoline to form gasohol.

FIG. 5 is a flow sheet denoting an alternate method to substantially absorb ethanol humidified carbon dioxide with gasoline to form gasohol.

FIG. 6 is a flow sheet denoting a method to substantially extract ethanol contained within water with gasoline to form gasohol.

### DETAILED DESCRIPTION OF THE PREFERRED EMBODIMENT

In the preferred embodiment of the present invention, a fermented broth is co-mingled with carbon dioxide, forming two phases for humidifying the carbon dioxide with ethanol from the fermented broth. The operating temperature range for fermentation is about 30° C. to about 35° C.

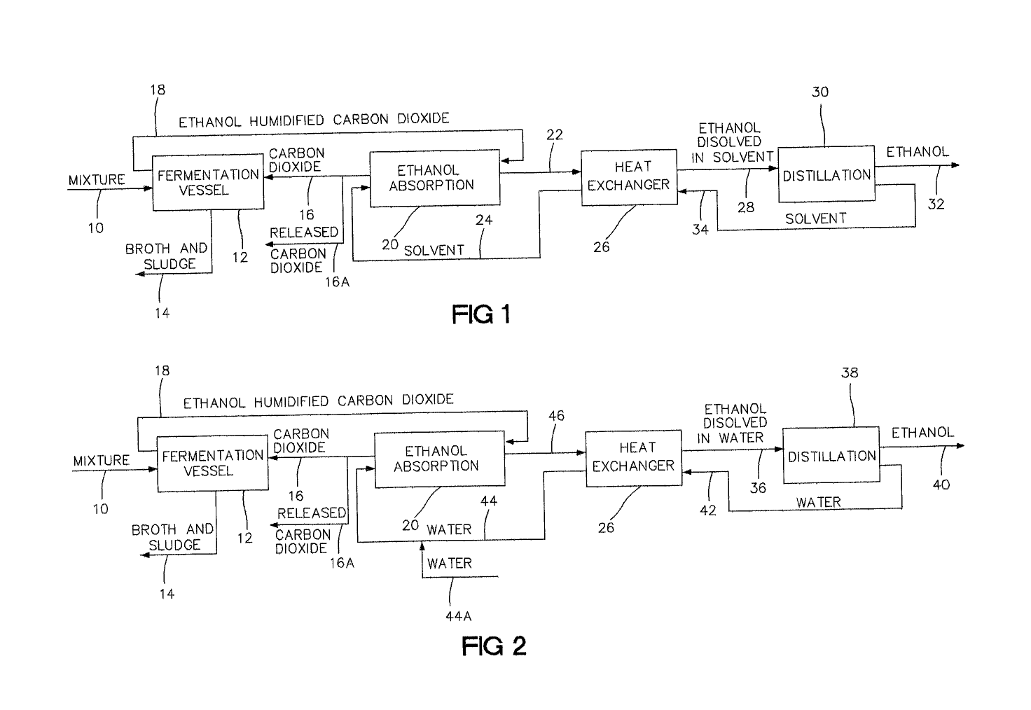

The flow diagram of FIG. 1 illustrates the general preferred embodiment of the present invention. In the diagram, rectangles represent stages, operations or functions of the present invention and not necessarily separate components. Arrows indicate direction of flow of material in the method.

Referring to FIG. 1, a mixture of microorganisms, nutrients and sugars 10 is provided to a fermentation vessel 12, to form a fermentation broth. Upon fermentation, the broth forms ethanol and carbon dioxide which is transmitted to provided carbon dioxide 16, devoid of released carbon dioxide 16A. Released carbon dioxide 16A removes the quantity of carbon dioxide formed from fermentation. The humidification and removal of formed ethanol contained within humidified carbon dioxide 18 is transported to ethanol absorption 20 which dissolves ethanol within a solvent 24 to form dissolved ethanol 22 which is then transmitted to heat exchanger 26 which, forms heated ethanol within solvent 28, and is then transported to distillation 30 to distill ethanol 32 from the solvent 34 for transfer to heat exchanger 26. Solvent 34, produced from distillation 30, is then transmitted to heat exchanger 26 which transfers heat to dissolved ethanol 22 to establish heated ethanol dissolved within solvent 28 for transfer to distillation 30 which provides solvent 24 for employment within ethanol absorption 20. Solvent 34 must be capable of dissolving ethanol and capable of separation of ethanol from distillation 30 to provide solvent substantially free of ethanol. The method will withdraw the ethanol humidified carbon dioxide, containing carbon dioxide from the fermentation, 18 from the fermentation vessel. Fermentation sludge and broth 14, removed from the fermentation vessel, is available for further treatment. The method depicted in FIG. 1 employs humidification for transmitting ethanol from fermented broth to carbon dioxide. Controlled flow rate of gaseous carbon dioxide is provided to maintain ethanol concentration within the broth. The humidified carbon dioxide may contain various volatile compounds from the fermented broth. The method can be operated either batch ways or by continuous operation. Microorganisms within the fermentation broth become inacive and are removed from the fermentation vessel as sludge and broth 14. The fermentation vessel 12, utilized in plug flow operation, will segregate microorganisms of reduced inactivity for removal from the fermentation vessel as sludge and broth 14. The mixture 10, containing microorganisms, nutrients and sugars can be a separate solution of microorganisms and a solution of nutrients and a solution of sugars combined within the fermentation vessel or a single solution containing microorganisms, nutrients and sugars. The sugars, capable of fermentation to produce ethanol and carbon dioxide, may consist of the group of carbohydrates which include glucose and xylose. Energy formed from fermentation must be removed from the broth to maintain broth temperature. Removal of the energy is commonly accomplished by a heat exchanger, not depicted in FIG. 1, to maintain substantially isothermal broth. Carbon dioxide 16, combined with carbon dioxide from fermentation, will percolate upward within fermentation vessel 12 for removal of ethanol humidified carbon dioxide 18. It is assumed that counter flow of carbon dioxide 16 is supplied from the lower end of the fermentation vessel 12 to remove ethanol humidified carbon dioxide 18. Ethanol absorption 20, from ethanol humidified carbon dioxide 18, is ordinarily a scrubbing tower supplied by a solvent to dissolve ethanol within a solvent to remove ethanol and form carbon dioxide 16. Microorganisms within the fermentation broth likely contain or include yeasts.

Referring to FIG. 2, a mixture of microorganisms, nutrients and sugars 10 is provided to a fermentation vessel 12, to form a fermentation broth. Upon fermentation, the broth forms ethanol and carbon dioxide which is transmitted to the provided carbon dioxide 16, devoid of released carbon dioxide 16A. Released carbon dioxide 16A removes the quantity of carbon dioxide formed from fermentation. The humidification and removal of formed ethanol contained within humidified carbon dioxide 18 is transported to ethanol absorption 20 which dissolves ethanol within water 44 to form dissolved ethanol 46 and is then transmitted to heat exchanger 26 which is then transported to distillation 38 to distill ethanol 40 from the water 42 for transfer to heat exchanger 26. Water 42 produced from distillation 38 is then transmitted to heat exchanger 26 which transfers heat to dissolved ethanol 46 is establish heated ethanol dissolved within water 36 for transfer to distillation 38 and produces water 44 for employment within ethanol absorption 20. The method will withdraw, from the fermentation vessel, ethanol humidified carbon dioxide 18 containing carbon dioxide from fermentation. Fermentation sludge and fermentation broth 14, removed from the fermentation vessel, is available for further treatment. Water 44 generally contains ethanol not stripped within distillation 38. Water 44A is added to water 44 to provide makeup for water removed within distilled ethanol 48. Ethanol absorption 20, from ethanol humidified carbon dioxide 18, is routinely a scrubbing tower supplied by water to dissolve ethanol within water to remove ethanol and form carbon dioxide 16.

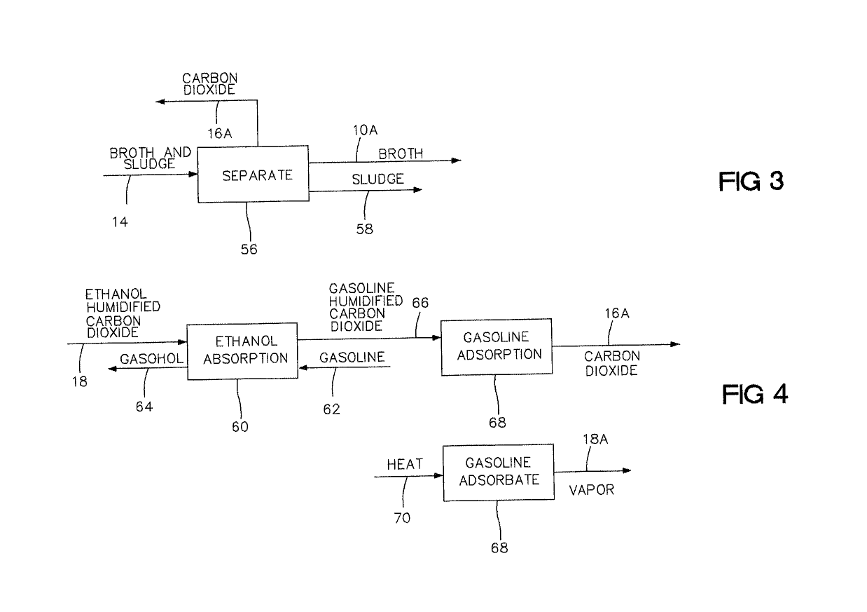

Referring to FIG. 3, broth and sludge 14 is transmitted to separate stage 56 which finctions to separate solution 10A from sludge 58. Separate stage 56, for example, can be supplied by a microfiltration filter or a settling tank. Broth 10A after separation is then recycled to the fermentation vessel 12 to regulate fermentation broth and combine with mixture 10. Broth and sludge 14 may be concentrated by microfiltration to reduce volume to separation stage 56. Carbon dioxide 16A, formed within separate stage 56, during fermentation is combined with humidified carbon dioxide 18. A mixture of microorganisms within the broth has been accordingly rendered insoluble.

Referring to FIG. 4 ethanol humidified carbon dioxide 18 is subjected to ethanol absorption 60 by gasoline 62 to dissolve ethanol and form gasohol 64 and gasoline humidified carbon dioxide 66 which is transmitted to gasoline adsorption 68 to substantially adsorb gasoline from carbon dioxide and produce carbon dioxide 16A substantially free of gasoline. Gasoline adsorption 68 becomes gasoline absorbate 68 and is heated by heat 70 to form vapor 18A. Adsorption media contained within gasoline adsorption 68 is reused to adsorb additional gasoline from gasoline humidified carbon dioxide 66.

Referring to FIG. 5 ethanol humidified carbon dioxide 18 is subjected to ethanol absorption 72 by gasoline 74 for absorption of ethanol to form gasohol 78 for transportation to distill stage 80 to form vapor 84 and gasohol 82. Vapor 84 is condensed in condense stage 86 to form a condensate 88 separated into upper phase 78A and a lower phase 92 in phase forming stage 90. The upper phase 78A is combined with gasohol 78. The lower phase 92 is transfered to distill stage 94 to produce vapor 98 and raffinate 96. Accordingly dehydrated gasohol 82 is produced. Raffinate 96, composed fundamentally of water is available for process water. Undisclosed within FIG. 5, gasoline humidified carbon dioxide 76 is subjected to additional treatment to free gasoline from carbon dioxide.

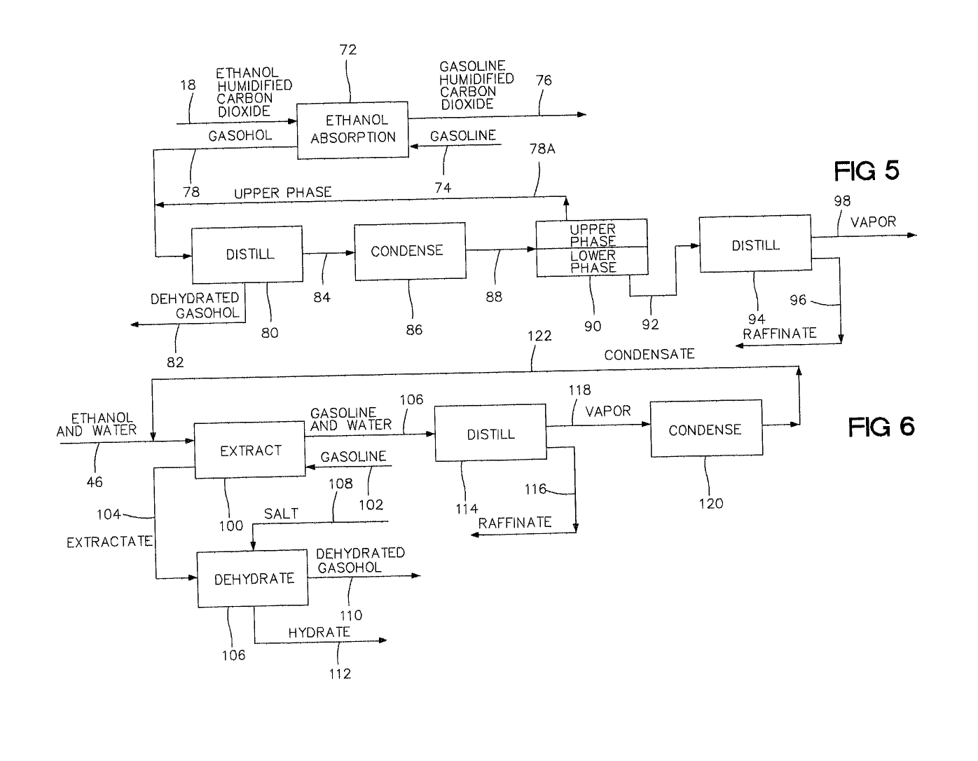

Referring to FIG. 6, water containing ethanol 46 is extracted by extract stage 100 with gasoline 102, to produce gasoline and water 106, and to produce an extractate 104 containing extracted ethanol. Gasoline and water 106 are distilled within distill stage 114 to produce raffinate 116 and vapor 118 to be condensed within condense stage 120 to produce condensate 122 to be combined with water containing ethanol 46. Extracxtate 104 is dehydrated within dehydrate stage 106 from salt 108 to produce gasohol 110 and a hydrate 112. Raffinate 116 is commonly used to supply water 44 and water 44A. Dehydration of gasohol, containing extracted ethanol, may be performed by a salt to form a hydrate or a concentrated solution such as calcium chloride or a dehydration desiccant, as an example, silica gel. Undisclosed within FIG. 6, Hydrate 112 is subjected to heat to produce a vapor for combination with vapor 118 and a desiccant for combination with salt 108. Accordingly, dehydrated gasohol 110 is produced. The following examples are set forth to illustrate more clearly the principles and practice of the invention.

### EXAMPLE 1

To demonstrate the method, 7 grams of Red Star yeast is dissolved within 50 cc of water to form a mixture. 10 grams of glucose is dissolved within 50 cc of water to form a separate mixture. Both mixtures are combined within a one quart jar, with stirring, and then covered to establish aerobic fermentation within the combined mixture. Fermentation, with occasional agitation, proceeds at room temperature until foaming and effervesce of carbon dioxide no longer emanates from the fermenting mixture. Fermentation is allowed to continue for 24 hours. The fermented mixture, thus formed, is allowed to settle to segregate sludge from the broth. The broth is decanted from the sludge to provide broth separated from the sludge.

### EXAMPLE 2, PART 1

To illustrate a method for producing anhydrous gasohol, one pint of gasohol contained within a one quart jar, is brought to boil. The boiling point, not definite, was initially found to be about 50 degrees C. The gasohol following investigation was discarded.

### EXAMPLE 2, PART 2

One pint of gasohol combined with a volume of water contained within a one quart jar, is stirred to saturate the gasohol with water and then decanted to separate saturated gasohol from the water.

### EXAMPLE 2, PART 3

One pint of gasohol saturated with water, contained within a one quart jar, is brought to boil. The boiling point, not definite, initially was found to be about 40 degrees C. indicating a low boiling ternary vapor of water, ethanol and a component of gasoline to yield anhydrous gasohol. The gasohol following investigation was discarded.

What is claimed is:

1. A method to separate ethanol from a fermentation broth, which comprises:
providing a fermentation vessel within which ethanol and carbon dioxide are produced, and
providing a mixture of microorganisms, nutrients and sugars to form a volume of broth contained within said fermentation vessel, and
subjecting said broth within said fermentation vessel to fermentation to form ethanol and carbon dioxide, and
providing a controlled flow rate of gaseous carbon dioxide to said fermentation vessel to humidify ethanol to regulate concentration of ethanol within the broth to between about 6% to about 12%, and separating the carbon dioxide, containing humidified ethanol and carbon dioxide produced by fermentation, from the fermentation vessel, and
removing means for separation of ethanol from the separated humidified carbon dioxide to substantially remove ethanol from carbon dioxide to provide carbon dioxide to humidify ethanol, and
separating sludge and broth from said fermentation vessel, and
providing said mixture, to replace the volume of separated sludge and broth, to maintain substantially constant volume of broth within the fermentation vessel thereby removing ethanol within broth, to regulate concentration of ethanol, and removing carbon dioxide from the fermentation vessel.

2. The method of claim 1 wherein said fermentation broth contains nutrients employed for fermentation substantially maintained to provide nutrients utilized within fermentation.

3. The method of claim 1 wherein said fermentation broth is established at a temperature and maintained at substantially isothermal conditions.

4. The method of claim 1 wherein said sugars, capable of fermentation within which ethanol and carbon dioxide are produced, are selected from the group consisting of glucose and xylose and mixtures thereof.

5. The method of claim 1 wherein said carbon dioxide, containing humidified ethanol and carbon dioxide produced by fermentation, contains ethanol vapor produced from heat formed during fermentation.

6. The method of claim 1 wherein the microorganisms are yeasts capable of forming enzymes required for fermentation to form ethanol and carbon dioxide.

7. The method of claim 1 wherein said fermentation vessel is operated in a continuous manner.

8. The method of claim 1 wherein said sludge and broth removed from said fermentation vessel are settled within a vessel to substantially separate broth from sludge.

9. The method of claim 8 wherein the broth separated from the sludge is combined with said mixture of microorganisms, nutrients and sugars to maintain volume of broth within said fermentation vessel.

10. The method of claim 1 wherein the microorganisms are capable of forming enzymes required for fermentation to form ethanol and carbon dioxide.

11. The method of claim 1 wherein said humidified carbon dioxide, containing ethanol, is scrubbed by a solvent to provide a solution containing ethanol and to provide carbon dioxide.

12. The method of claim 1 wherein said humidified carbon dioxide, containing ethanol, is scrubbed by water to provide a solution containing ethanol and to provide carbon dioxide humidified by water.

13. The method of claim 12 wherein the solution containing ethanol is extracted by gasoline to produce an extactate of gasoline within dissolved ethanol and a solution substantially free of ethanol.

14. The method of claim 13 wherein the extractate is substantially dehydrated.

15. The method of claim 13 wherein the solution substantially free of ethanol is distilled to produce vapor and a raffinate.

16. The method of claim 1 wherein the carbon dioxide is humidified and saturated by water so that further humidification by the carbon dioxide will produce humidified ethanol from the fermentation broth without substantially producing humidified water from the fermentation broth.

17. The method of claim 1 wherein said humidified carbon dioxide, containing ethanol, is scrubbed by gasoline to provide gasohol containing ethanol and to provide carbon dioxide containing gasoline.

18. The method of claim 17 wherein the gasohol containing water is dehydrated by forming a hydrate and dehydrated gasohol.

19. The method of claim 17 wherein the gasohol containing water is dehydrated by distillation forming an azeotrope and dehydrated gasohol.

20. The method of claim 17 wherein the carbon dioxide containing gasoline is subjected to adsorption to form carbon dioxide substantially free of gasoline and an absorbate containing gasoline.
